(19) United States
(12) Patent Application Publication
(10) Pub. No.: US 2012/0220732 A1
(43) Pub. Date: Aug. 30, 2012

(54) CURING ACTIVATORS

(75) Inventors: Salvatore Cotugno, Roma (IT); Paolo Straffi, Roma (IT); Barbara Secchi, Roma (IT)

(73) Assignee: BRIDGESTONE CORPORATION, Chuo-Ku, Tokyo (JP)

(21) Appl. No.: 13/390,041

(22) PCT Filed: Aug. 9, 2010
(86) PCT No.: PCT/IB2010/001971

§ 371 Date: Apr. 27, 2012

(30) Foreign Application Priority Data
Aug. 10, 2009 (IT) TO2009A000631

(51) Int. Cl. C08L 9/06; C07C 53/126

(52) U.S. Cl. 525/366; 562/553

(57) ABSTRACT

Use of compounds of molecular formula (I) as curing activators of mixes including a cross-linkable, unsaturated-chain polymer base; (R₁CONR₂CHR₃COO⁻)n Xⁿ⁺ (I) where: R¹ is an aliphatic group C₆-C₂₃, R₂ is H or an aliphatic group C₁-C₈, R₃ is H or an aliphatic or aromatic group C₁-C₈, X is a metal cation, and n is an integer from 1 to 3.

### TECHNICAL FIELD

The present invention relates to curing activators.

### BACKGROUND ART

Depending on the requirements involved, rubber mix curing normally calls for special additives, such as curing activators and accelerating agents.

In some industrial applications, curing must be performed rapidly, so particularly effective accelerating agents, known as “boosters”, are employed.

In recent times, however, use of some of these booster accelerating agents has been strictly limited for health reasons.

Commonly used curing activators are fatty acids, which, though effectively activating the curing process, may pose problems in terms of adhesion of the green mix, by reacting with zinc oxide and producing zinc salts, which tend to migrate towards the surface of the mix.

New curing accelerating systems are therefore required, which provide for fast curing, while at the same time limiting the use of fatty acids.

### DISCLOSURE OF INVENTION

One object of the present invention is the use of compounds of molecular formula (I) as curing activators of mixes comprising a cross-linkable, unsaturated-chain polymer base;

(R₁CONR₂CHR₃COO⁻)nXⁿ⁺ (I)

where:

R₁ is an aliphatic group C₆-C₂₃,

R₂ is H or an aliphatic group C₁-C₈,

R₃ is H or an aliphatic or aromatic group C₁-C₈,

X is a metal cation, and

n is an integer from 1 to 3.

The aliphatic group R₁ preferably comprises a double bond.

The curing activator preferably has a molecular formula in the group comprising:

CH₃(CH₂)₇CHCH(CH₂)₇CONHCH₂COO⁻X⁺;

and

CH₂CH(CH₂)₈CONHCH₂COO⁻X⁺.

Another object of the present invention is a mix comprising a cross-linkable, unsaturated-chain polymer base, characterized by comprising as a curing activator a compound of molecular formula (I).

Another object of the present invention is a rubber product, characterized by being made from a mix comprising a compound of molecular formula (I) as a curing activator.

Another object of the present invention is a tyre, characterized by comprising at least one rubber part made from a mix comprising a compound of molecular formula (I) as a curing activator.

The following are purely non-limiting examples, for a clearer understanding of the present invention.

### EXAMPLES

In the examples described below, two different compounds (a, b) in the class of curing aids according to the present invention were employed.

The two curing aids used were:
compound (a) of molecular formula CH₃ (CH₂) ₇CHCH (CH₂) ₇CONHCH₂COO⁻Na⁺; and
compound (b) of molecular formula CH₂CH (CH₂) ₈CONHCH₂COO⁻Na⁺.

Purely by way of example, the above curing activators were tested with TBBS and DPG accelerating agents, as described below.

### TBBS Mixes

Four mixes (A₁, A₂, B₁, B₂) were prepared, each comprising one of the above two activators (a, b) according to the present invention. More specifically, the two different activators (a, b) were used in two different concentrations.

Table I shows the compositions in phr of the above mixes.

TABLE I

| | | | |
|---|---|---|---|
| A₁ | A₂ | B₁ | B₂ |

| | |
|---|---|
| S-SBR | 100 |
| N660 | 40 |
| ZnO | 2 |
| S | 2 |
| TBBS | 2 |

| | | | | |
|---|---|---|---|---|
| Comp. (a) | 2 | 5 | | |
| Comp. (b) | | | 2 | 5 |

To accurately assess the advantages of using the curing activators according to the present invention, a control mix (MC_{TBBS}) was prepared comprising stearic acid as an activating fatty acid.

Table II shows the composition in phr of the control mix.

TABLE II

| | MC_{TBBS} |
|---|---|
| S-SBR | 100 |
| N660 | 40 |
| Stearic acid | 1 |
| ZnO | 2 |
| S | 2 |
| TBBS | 2 |

### DPG Mixes

Four mixes (A₃, A₄, 13₃, B₄) were prepared, comprising the curing activator (a) and curing activator (b) according to the present invention respectively. More specifically, as shown below, curing activators (a) and (b) were tested in both the presence and absence of fatty acids.

To accurately assess the advantages of using the curing activators according to the present invention, a control mix (MC_{DPG}) was prepared comprising fatty acids as activators.

Table III shows the compositions in phr of mixes A₃, A₄, B₃, B₄ and control mix MC_{DPG}.

TABLE III

| | A₃ | A₄ | B₃ | B₄ | MC_{DPG} |
|---|---|---|---|---|---|
| S-SBR | 100 | | | | |
| N660 | 40 | | | | |
| Fatty acids | | 1 | | 1 | 1 |
| ZnO | 2 | | | | |
| S | 1 | | | | |
| DPG | 1 | | | | |
| Comp. (a) | 1 | 1 | | | |
| Comp. (b) | | | 1 | 1 | |

### Testing

The mixes with the above compositions were curing tested at different temperatures. More specifically, the rheometric properties of each mix were tested according to ISO Standard 6502.

Table IV shows the rheometric property results of the TBBS mixes. The curing tests were performed at temperatures of 145° C., 160° C., 175° C., and 195° C. The MH and ML values are expressed in dNm, and T′10 and T′90 in minutes.

TABLE IV

| | | | | |
|---|---|---|---|---|
| MC_{TBBS} | A₁ | A₂ | B₁ | B₂ |

145° C.

| | | | | | |
|---|---|---|---|---|---|
| ML | 1.81 | 2.02 | 2.05 | 1.95 | 1.96 |
| MH | 22.03 | 21.06 | 20.37 | 20.96 | 19.74 |
| T10 | 26.39 | 13.28 | 9.96 | 14.6 | 9.68 |
| T90 | 37.64 | 24.21 | 21.59 | 25.45 | 19.71 |

160° C.

| | | | | | |
|---|---|---|---|---|---|
| ML | 1.72 | 1.76 | 1.76 | 1.72 | 1.73 |
| MH | 21.23 | 21.35 | 20.66 | 20.83 | 19.26 |
| T10 | 8.79 | 4.52 | 3.34 | 4.96 | 3.39 |
| T90 | 14.9 | 11.12 | 10.78 | 11.27 | 9.54 |

175° C.

| | | | | | |
|---|---|---|---|---|---|
| ML | 1.53 | 1.51 | 1.54 | 1.52 | 1.53 |
| MH | 21.24 | 21.38 | 20.78 | 20.73 | 19.55 |
| T10 | 3.23 | 1.54 | 1.18 | 1.8 | 1.29 |
| T90 | 7.09 | 5.44 | 5.91 | 5.69 | 5.6 |

195° C.

| | | | | | |
|---|---|---|---|---|---|
| ML | 1.34 | 1.4 | 1.45 | 1.37 | 1.36 |
| MH | 20.77 | 20.61 | 19.83 | 20.13 | 18.72 |
| T10 | 0.94 | 0.54 | 0.45 | 0.6 | 0.5 |
| T90 | 2.28 | 2.14 | 2.31 | 2.25 | 2.39 |

Table V shows the rheometric property results of the DPG mixes. The curing tests were performed at a temperature of 160° C.

Because curing did not take place in the conditions set, Table V does not show the results of the control mix MC_{DPG}.

TABLE V

| | A₃ | A₄ | B₃ | B₄ |
|---|---|---|---|---|
| ML | 1.67 | 1.52 | 1.77 | 1.57 |
| MH | 16.89 | 12.41 | 17.53 | 14.57 |
| T10 | 1.45 | 1.5 | 1.31 | 1.45 |
| T50 | 4.11 | 4.7 | 3.75 | 6.75 |
| T90 | 14.87 | 18.43 | 14.45 | 18.99 |

As the above results clearly show, the curing activators according to the present invention provide for a surprisingly significant increase in the accelerating power of the accelerating agents used with them. This constitutes a major advantage in terms of production, as well as a valid alternative to certain “booster” accelerating agents widely used in the rubber industry and currently under investigation by health protection agencies.

The curing activators according to the present invention also surprisingly provide for eliminating use of fatty acids in the mix as activators, which has the advantage of reducing the number of components in the mix, as well as solving the adhesion problems posed by the salts, produced by reaction of the fatty acids with zinc oxide, migrating towards the surface of the mix.

As will be clear to anyone skilled in the art, the present invention may be used to advantage in industries involving the manufacture of fast-cured rubber products, in particular, and preferably, the tyre industry.

1. Use of compounds of molecular formula (I) as curing activators of mixes comprising a cross-linkable, unsaturated-chain polymer base;
(R₁CONR₂CHR₃COO⁻)nXⁿ⁺ (I)
where:
R₁ is an aliphatic group C₆-C₂₃,
R₂ is H or an aliphatic group C₁-C₈,
R₃ is H or an aliphatic or aromatic group C₁-C₈,
X is a metal cation, and
n is an integer from 1 to 3.

2. Use of compounds of molecular formula (I) as claimed in claim 1, characterized in that the aliphatic group R₁ comprises a double bond.

3. Use of compounds of molecular formula (I) as claimed in claim 2, characterized by having at least one of molecular formula comprised in the group consisting of:
CH₃(CH₂)₇CHCH(CH₂)₇CONHCH₂COO⁻X⁺; and
CH₂CH(CH₂)₈CONHCH₂COO⁻X⁺.

4. Use of compounds of molecular formula (I) as claimed in claim 1, characterized in that X⁺ is Na⁺.

5. A mix comprising a cross-linkable, unsaturated-chain polymer base, characterized by comprising as curing activator a compound of molecular formula (I) as claimed in claim 1.

6. A mix according to claim 5, characterized by comprising 0.5 to 10 phr of the curing activator.

7. A rubber product, characterized by being made from a mix as claimed in claim 5.

8. A tyre, characterized by comprising at least one rubber part made from a mix as claimed in claim 5.
